(19) United States
(12) Patent Application Publication
(10) Pub. No.: US 2002/0177225 A1
(43) Pub. Date: Nov. 28, 2002

(54) Bio-screening methods and biomaterial thereof

(76) Inventors: Rodolfo C. Quijano, Laguna Hills, CA (US); Hosheng Tu, Tustin, CA (US)

Correspondence Address: Hosheng Tu, 15 Riez, Newport Coast, CA, 92657, US

(21) Appl. No.: 09/865,251

(22) Filed: May 25, 2001

(51) Int. Cl. A61K 45/00; C12N 5/06

(52) U.S. Cl. 435/325; 424/93.7

(57) ABSTRACT

This invention discloses a bio-screening method for preparing a biomaterial adapted for medical device use, the method comprising chemically treating tissue of biological origin from a mammal. The mammal of the present invention comprises a perissodactylous ungulate that is naturally absent of foot-and-mouth disease and/or transmissible spongiform encephalopathy disease.

### TECHNICAL FIELD OF INVENTION

The present invention generally relates to methods of preparing tissue of biological origin as biomaterial for medical device use, and more particularly, to such methods for bio-screening available tissue from animals and biomaterials therefrom that are absent of transmissible spongiform encephalopathy and/or foot-and-mouth diseases.

### BACKGROUND OF THE INVENTION

Foot-and-mouth disease (FMD) is a highly contagious illness of cloven-hoofed animals, especially cattle, sheep, goats and pigs. The virus is now known to be a member of the picornavirus family, affecting over 100,000 animals a year and resulting in significant economic loss. It belongs to the aphthovirus genus, which is composed of small (diameter of 25 to 30 nm) acid-labile RNA virus containing naked icosahedral nucleocapsids (Wilfert C M, in Cecil Textbook of Medicine, vol.2, 16^{th} edition, pp. 1643-1644, published by W. B. Sanders, 1982). The disease in animals is characterized by fever and increased salivation, with the appearance of vesicular lesions on the mucous membranes of the mouth and tongue, while the V antigens are not detectable until approximately the tenth day of infection and persist for years.

Foot-and-mouth disease occurs in many areas of the world outside the United States where vaccination programs have been largely effective. In general, the prognosis of infection with mumps virus is excellent. Mumps is a self-limited infection, and there is no specific therapy available. Mason et al. in U.S. Pat. No. 5,612,040 discloses a vaccine for the protection of susceptible animals against foot-and-mouth disease, wherein the vaccine comprises a mutant virus from which a special group of amino acid sequence has been replaced and the mutant virus retains its antigenicity but is not infectious. But the biomaterial adapted for medical device use of biological origin from a mammal cannot rely on the animals that have been vaccinated due to its safety concern. The biomaterial should come from the perissodactylous ungulate that is naturally absent of foot-and-mouth disease.

Bovine spongiform encephalopathy (BSE) or “mad cow disease” appears to have originated from scrapie, an endemic spongiform encephalopathy of sheep and goats that has been recognized in Europe since the mid-18^{th} century. It has since spread to most sheep-breeding countries and is widespread in the United Kingdom (UK), which has affected nearly 200,000 cattle. It leaves in its wake an outbreak of human Creutzfeldt-Jakob disease, most probably resulting from the consumption of beef products contaminated by central nervous system tissue.

Until 1988 the rendered carcasses of livestock (including sheep) were fed to ruminants and other animals as a protein-rich nutritional supplement. During rendering, carcasses from which all consumable parts had been removed were milled and then decomposed in large vats by boiling at atmospheric or higher pressures, producing an aqueous slurry of protein under a layer of fat (tallow). After the fat was removed, the slurry was desiccated into a meat and bone meal product that was packaged by the animal food industry and distributed to owners of livestock and other captive animals. The fat is used in an amazing array of products such as soap, lipstick, linoleum and glue.

BSE is not restricted to the UK. Cases have occurred in many other countries as a result of imported live animals or livestock food supplements. In some countries, including the UK, the incidence of new cases is decreasing, but in other countries—Belgium, Denmark, France, Portugal, Germany, Italy, Liechtenstein, Switzerland, Spain, the Netherlands, and Ireland—the incidence appears to be increasing. A contributing factor to this may be new infections from contaminated feed intended for other species of cloven-hoofed mammals. Although the contaminated feed is cooked, the BSE prion can survive.

Prions are infectious pathogens that cause central nervous system spongiform encephalopathies in humans and animals. Prions are distinct from bacteria, viruses and viroids. The predominant hypothesis at present is that no nucleic acid component is necessary for infectivity of prion protein. Further, a prion which infects one species of animal (e.g. human) may not infect another (e.g., a mouse) under generally normal conditions.

The human prion diseases (Mastrianni J A, et al. Sem Neurology 20(3):337-352, 2000) include kuru, Creutzfeldt-Jakob disease (CJD), Gerstmann-Straussler-Scheinker (GSS) syndrome, fatal insomnia (FI), and variant/new variant CJD (vCJD). In addition to these human diseases, prion-related diseases have been recognized in several animal hosts. Scrapie is a naturally occurring disease of sheep and goats that causes ataxia, behavioral changes, and a severe pruritus that leads to scraping behavior, from which the disease was named. Additional prion diseases in animals include transmissible mink encephalopathy (TME), chronic wasting disease (CWD) of mule, deer and elk, feline spongiform encephalopathy (FSE), and bovine spongiform encephalopathy (BSE), among others. CWD is one of the transmissible spongiform encephalopathies (TSE) that appears to be a naturally occurring disease of North America.

Within weeks of identification of the first case of BSE, concern was expressed about human risk, and as the epidemic unfolded, a series of measures was taken to eradicate BSE and prevent potentially infected tissues from reaching the human food chain or human biomedical use. Another concern was the discovery that some exotic zoo ungulates, as well as domestic and captive wild cats, were becoming infected. The ungulates and domestic cats had also been fed diets supplemented by meat and bone meal, and the wild cats had been fed uncooked tissues, including cattle heads and spines. The possibility could therefore not be ignored that the disease might also cross the species barrier to humans from the consumption of beef or dairy products, or perhaps from occasional contact with cattle by ranchers, dairyman, or slaughterhouse workers, among other routes.

A similarity between kuru and scrapie was noticed that leads to a hypothesis of the initial transmission of CJD to experimental primates. It was conceived that extracts prepared from patients dying of kuru being inoculated into non-human primates might transmit the disease after a prolonged incubation period. Gajdusek et al. demonstrated the transmissibility of kuru to chimpanzees after incubation periods ranging from 19 to 21 months (Gajdusek et al. Nature 209:794-796 (1966). Over the last 25 years, about 300 cases of CJD and kuru have been transmitted to a variety of apes and monkeys. This raises the question that inter-species transmission among primates, including humans, is possible.

What muted concerns about human infection was the presumption that BSE originated from scrapie, and scrapie was not a human pathogen. Nevertheless, transmissible spongiform encephalopathy (TSE) may cause a spontaneous mutation in cattle. Experimental studies of species susceptibility to this new strain of mutated TSE had not sufficiently advanced to predict that humans would not be susceptible. The link has been established in laboratory studies showing identical, distinctive biological and molecular biological features of the pathologic agent isolated from BSE-infected cattle and human cases of varied Creutzfeldt-Jakob disease (vCJD). The source of contamination appears to have been beef. Contamination could have occurred in any of the following ways: cerebral vascular emboli from cranial stunning instruments used to immobilize cattle before killing by exsanguination; contact of muscle with brain or spinal cord tissue by saws or other tools used during slaughter; inclusion of paraspinal ganglia in cuts of meat containing vertebral tissue; and perhaps most importantly, the presence of residual spinal cord and paraspinal ganglia tissue in the paste of “mechanically recovered meat” that could legally be added previously to cooked meat products such as meat pies, beef sausages, and various canned meat preparations.

Presently there is no pharmacological treatment for the prion diseases, and symptomatic management is the course of action. Further, there is no test to detect the presence of the disease in live animal, while the incubation period of the TSE disease may last from 2 to 8 years. The prion is extremely resistant to heat, ultraviolet light, ionization radiation, and to normal sterilization processes. It does not result in a detectable immune response or inflammatory reaction in the animals carrying the disease.

The biomaterial adapted for medical device use of biological origin from a mammal cannot rely on the animals that has been vaccinated due to its safety concern. The biomaterial should come from the perissodactylous ungulate that is absent of foot-and-mouth disease. Though it has not been well established that a biomaterial prepared from tissue originated from a TSE or foot-and-mouth disease infected artiodactylous ungulates could transmit the disease to humans, a bio-screening means for only utilizing biomaterial from the perissodactylous ungulate that is absent of foot-and-mouth disease and/or absent of transmissible spongiform encephalopathy disease is warranted and desired.

### SUMMARY OF THE INVENTION

In general, it is an object of the present invention to provide a method for bio-screening and preparing a biomaterial adapted for medical device use, the method comprising chemically treating tissue of biological origin from a mammal, wherein the mammal excludes artiodactylous ungulates. In one embodiment, the artiodactylous ungulates that are excluded as a biomaterial of the present invention may include, not limited to, pig, hog, javelina, hippopotamus, camel, llama, mouse deer, giraffe, okapi, deer, pronghom, antelope, cattle, goat, sheep and the like. The artiodactylous ungulates might generally be susceptible to foot-and-mouth disease or transmissible spongiform encephalopathy disease.

It is another object of the present invention to provide a method for bio-screening and preparing a biomaterial adapted for medical device use, the method comprising chemically treating tissue of biological origin from a mammal, wherein the mammal comprises a perissodactylous ungulate. In one preferred embodiment, ungulate is absent of transmissible spongiform encephalopathy disease or absent of foot-and-mouth disease. The tissue that could be used as a biomaterial of the present invention include the source from a perissodactylous ungulate, wherein the perissodactylous ungulate may be selected from a group consisting of primate, rhino, tapir, zebra, and horse, among others.

It is a further object of the present invention to provide a biomaterial adapted for medical device use comprising tissue of biological origin from a mammal, wherein the mammal comprises a perissodactylous ungulate, and wherein the tissue may be selected from a group consisting of blood vessels, cardiac tissue valves, tendons, ligaments, venous valves, valved conduits, pericardia, fasciae, dura matter, epidermis, nerves, and biological membranes. In another embodiment, the medical device of the present invention may comprise a tissue bioprosthetic valve, a biological vascular graft, a pericardial valve, a biological venous valve, or the like, while the chemically treating method may comprise aldehydes or epoxy compounds as a second safeguard in the bio-screening process of the present invention.

### DETAILED DESCRIPTION OF EXEMPLARY EMBODIMENTS

BSE (bovine spongiform encephalopathy) is a fatal disease that causes progressive neurological degeneration in cattle. Similar to BSE, Creutzfeldt-Jakob disease (CJD) is a rare disease that occurs in humans. In 1966, following outbreaks of BSE among British cattle, scientists found a possible link between BSE and a new variant of CVD (vCJD). While it is not certain how BSE may be spread to humans, evidence indicates that humans may acquire vCJD after consuming BSE-contaminated cattle products. The diseases are invariably fatal; there is no known treatment or cure. To safeguard a patient from acquiring the BSE, it is one embodiment of the present invention to bio-screen and select the tissue as a biomaterial from naturally non-BSE infective mammals, such as a perissodactylous ungulate.

Both the new variant CJD and the classic CJD are slow degenerative diseases of the central nervous system whose symptoms include dementia and loss of motor skills. Classic CJD occurs sporadically worldwide at a rate of approximately one case per 1 million people per year, and the new vCJD is even more rare. CJD and vCJD are best thought of as two different diseases. The brain lesions of victims of classic CJD and vCJD are distinct. To date, vCJD has caused disease in younger patients and the mean duration of illness is more prolonged.

The source of BSE outbreak is uncertain, but it is thought to have been amplified by feeding cattle with meat-and-bone meal (MBM) from BSE-infected cattle. The USDA's Animal and Plant Health Inspection Service (APHIS) enforces explicit import regulations covering animals and animal products offered for import into the United States to prevent the importation of foreign exotic diseases such as BSE, foot-and-mouth disease, rinderpest and African swine fever. Due to absence of detective methods available in a live animal and long incubation periods of the disease in animals, it is one embodiment of the present invention to bio-screen the tissue to be used as a biomaterial for medical device use as a first safeguard to human health. It is another embodiment of the present invention to chemically treat the bio-screened tissue to render the finished biomaterial suitably adapted for medical device use; this serves as a second safeguard to human health.

It is documented (Wilfert C M, in Cecil Textbook of Medicine, vol.2, 16^{th} edition, pp. 1643-1644, published by W. B. Sanders, 1982) that foot-and-mouth disease is a highly contagious illness of cloven-hoofed animals. It is further reported (Factsheet—Veterinary Services, published by USDA APHIS, January 2001) that there is no evidence that BSE spreads horizontally, i.e. by contact between unrelated adult cattle or from cattle to other species. However, limited research strongly suggests that maternal or vertical transmission may occur at a very low level. It is, therefore, one embodiment of the present invention to bio-screen and prepare a biomaterial adapted for medical device use, the method comprising chemically treating tissue of biological origin from a mammal, wherein the mammal comprises a perissodactylous ungulate that is naturally absent of foot-and-mouth disease or transmissible spongiform encephalopathy disease.

“Odd-toed” or “odd-hoofed” mammals make up the perissodactyls while artiodactyls are “even-toed” ungulates. Unlike artiodactyls, perissodactyls either walk on three toes or on a single toe. The living perissodactylous ungulates may include primates, rhinos, tapirs, zebras, horses, and the like. From literature, the perissodactylous ungulate is generally absent of foot-and-mouth disease and/or absent of transmissible spongiform encephalopathy disease, which is suitably adapted as a tissue source for biomaterial of the present invention. The “biomaterial” is generally defined in this invention as a biocompatible material that is suitably adapted for medical use or as a constituent of the finished medical devices.

Artiodactyls are even-toed ungulates. This order of herbivorous mammals includes cloven-hoofed animals with two hoofs on each foot (like pigs) or four hoofs on each foot (like deer and cows). Artiodactyls (downloaded from www.EnchantedLearning.com) are divided into 3 suborders, 9 families and about 210 species, including pig, hog, javelina, hippopotamus, camel, llama, mouse deer, giraffe, okapi, deer, pronghorn, antelope, cattle, goat, and sheep, among others. From literature, the artiodactylous ungulate is generally susceptible of foot-and-mouth disease and/or susceptible of transmissible spongiform encephalopathy disease, which is excluded as a tissue source for biomaterial of the present invention in the bio-screening process.

Many medical materials used in the treatment of cardiovascular diseases are required to possess biocompatible and hemo-compatible properties without antigenicity. One method to treat tissue so as to render the tissue more suitable as a biomaterial is a process called chemical treatment. Several chemical treatment agent and methods have been disclosed. Among them, aldehydes (glutaraldehyde, formaldehyde, dialdehyde starch and the like), epoxy compounds, genipin, and their analog or derivatives thereof are all applicable in the bio-screening method of the present invention. Chemical treatment conditions and procedures to render the tissue suitable as a biomaterial depend on the property of each tissue and intended medical applications, wherein the conditions/procedures are well documented in published literature and well known to one who is skilled in the art.

Noishiki et al. in U.S. Pat. No. 4,806,595 discloses a novel method for preparing medical materials by using epoxy compounds as chemical treatment agent for tissue, the entire contents of which are incorporated herein by reference. The “epoxy compounds” as disclosed in the present invention include, but not limited to, glycol diglycidyl ether, polyol polyglycidyl ether, dicarboxylic acid diglycidylester, the analog, and derivatives thereof.

BSE has not been diagnosed in the United States and USDA has worked proactively to keep it that way. However, the earlier evidence of a potential link between BSE and vCJD prompted the introduction of a specific bovine offal (SBO) ban at slaughterhouse in 1989. The SBO ban excluded from human consumption of brain, spinal cord and other organs with potential BSE infectivity. It is one embodiment of the present invention to provide a biomaterial for medical device use, wherein the source of biomaterial excludes the brain, spinal cord and other organs with potential BSE infectivity. More specifically, the tissue of biological origin from artiodactylous ungulates with potential BSE infectivity is excluded as a biomaterial of the present invention. On the other hands, the present invention comprises the tissue of biological origin from a perissodactylous ungulate without naturally potential BSE infectivity.

The biomaterial adapted for medical device use of the present invention comprises tissue of biological origin from a mammal, wherein the mammal comprises a perissodactylous ungulate that is naturally absent of foot-and-mouth disease and/or transmissible spongiform encephalopathy disease. The raw material source or tissue may comprise blood, blood components, bones, blood vessels, cardiac tissue valves, tendons, ligaments, venous valves, valved conduits, pericardia, fasciae, dura matter, epidermis, nerves, biological membranes, collagen-containing material, and their derivatives thereof.

As an additional third safeguard for patient's health, the medical device made from biomaterial of the present invention is shaped and/or configured using a non-contacting element for trimming, cutting, tearing, drilling, and the like. The purpose of the non-contacting element is to prevent cross-contamination, particularly for the tissue originated from artiodactylous ungulates or contaminated perissodactylous ungulates. One example of the non-contacting element is a laser knife or laser fiber optic instrument.

From the foregoing description, it should now be appreciated that a bio-screening method for preparing a biomaterial and biomaterial therefrom adapted for medical device use or other biomedical applications, the method comprising chemically treating tissue of biological origin from a mammal, wherein the mammal comprises a perissodactylous ungulate that is naturally absent of foot-and-mouth disease and/or transmissible spongiform encephalopathy disease have been disclosed. While the invention has been described with reference to a specific embodiment, the description is illustrative of the invention and is not to be construed as limiting the invention. Various modifications and applications may occur to those who are skilled in the art, without departing from the true spirit and scope of the invention, as described by the appended claims.

What is claimed is:

1. A method for preparing a biomaterial adapted for medical device use, the method comprising chemically treating tissue of biological origin from a mammal, wherein the mammal excludes artiodactylous ungulates.

2. The method according to claim 1, wherein the artiodactylous ungulates are selected from a group consisting of pig, hog, javelina, hippopotamus, camel, llama, mouse deer, giraffe, okapi, deer, pronghom, antelope, cattle, goat, and sheep.

3. A biomaterial adapted for medical device use comprising tissue of biological origin from a mammal, wherein the mammal comprises a perissodactylous ungulate.

4. The biomaterial according to claim 3, wherein the perissodactylous ungulate is absent of foot-and-mouth disease.

5. The biomaterial according to claim 4, wherein the perissodactylous ungulate is selected from a group consisting of primate, rhino, tapir, zebra, and horse.

6. The biomaterial according to claim 3, wherein the perissodactylous ungulate is absent of transmissible spongiform encephalopathy disease.

7. The biomaterial according to claim 6, wherein the perissodactylous ungulate is selected from a group consisting of primate, rhino, tapir, zebra, and horse.

8. The biomaterial according to claim 3, wherein the medical device is configured or shaped using a non-contacting element.

9. The biomaterial according to claim 3, wherein the medical device is a tissue bioprosthetic valve.

10. The biomaterial according to claim 3, wherein the medical device is a bioprosthetic vascular graft.

11. The biomaterial according to claim 3, wherein the medical device is a chemically treated tissue of said biological origin.

12. The biomaterial according to claim 11, wherein the tissue is treated in an aldehyde solution or in an epoxy compounds solution.

13. The biomaterial according to claim 11, wherein the tissue is selected from a group consisting of blood components, blood vessels, cardiac tissue valves, tendons, ligaments, venous valves, valved conduits, pericardia, fasciae, dura matter, epidermis, nerves, and biological membranes.

14. A method for preparing a biomaterial adapted for medical device use, the method comprising chemically treating tissue of biological origin from a mammal, wherein the mammal comprises a perissodactylous ungulate.

15. The method according to claim 14, wherein the perissodactylous ungulate is absent of foot-and-mouth disease.

16. The method according to claim 15, wherein the perissodactylous ungulate is selected from a group consisting of primate, rhino, tapir, zebra, and horse.

17. The method according to claim 14, wherein the perissodactylous ungulate is absent of transmissible spongiform encephalopathy disease.

18. The method according to claim 17, wherein the perissodactylous ungulate is selected from a group consisting of primate, rhino, tapir, zebra, and horse.

19. The method according to claim 14, wherein the tissue is chemically treated by a chemical solution selected from a group consisting of aldehydes and epoxy compounds.

20. The method according to claim 14, wherein the tissue is selected from a group consisting of blood components, blood vessels, cardiac tissue valves, tendons, ligaments, venous valves, valved conduits, pericardia, fasciae, dura matter, epidermis, nerves, and biological membranes.
